(19) United States
(12) Patent Application Publication
(10) Pub. No.: US 2009/0314720 A1
(43) Pub. Date: Dec. 24, 2009

(54) NOVEL COMPOSITIONS AND USES THEREOF

(76) Inventors: Desmond Smith, Swindon (GB); Colin Smith, Swindon (GB); Duncan Watson, Staines (GB)

Correspondence Address: MCANDREWS HELD & MALLOY, LTD, 500 WEST MADISON STREET, SUITE 3400, CHICAGO, IL, 60661, US

(21) Appl. No.: 12/474,355

(22) Filed: May 29, 2009

Related U.S. Application Data
(60) Provisional application No. 61/057,637, filed on May 30, 2008.

(30) Foreign Application Priority Data
May 30, 2008 (GB) 0809912.9

(51) Int. Cl. C02F 1/52; C02F 5/08

(52) U.S. Cl. 210/702; 252/175

(57) ABSTRACT

The present technology provides systems and methods for treating a fluid by contacting a compound to the fluid. The compound comprises an azodicarbonamide or an azodicarbonamide derivative, wherein the treatment reduces the amount of hydrogen sulphide, iron sulphide and/or mercaptan in the fluid. Certain embodiments of the present technology provide a composition comprising an azodicarbonamide or an azodicarbonamide derivative and a support and/or a suspension agent.

### RELATED APPLICATIONS

This non-provisional application claims priority to (1) U.S. Provisional Application No. 61/057637, filed on May 30, 2008; and (2) Great Britain Application No. 0809912.9, filed May 30, 2008. The present application hereby incorporates U.S. application Ser. No. 61/057637 and Great Britain Application No. 0809912.9 in their entireties.

### FIELD OF THE INVENTION

The present technology relates to chemical compositions for reducing the levels of one or more of hydrogen sulphide, iron sulphide, or mercaptans in a fluid, for example in a fluid stream, for example a hydrocarbon stream (such as crude oil or natural gas) or water stream.

### BACKGROUND OF THE INVENTION

It is desirable to remove hydrogen sulphide or mercaptan (thiol) impurities from natural gas, crude oil, other hydrocarbon fluids or fluid streams, water etc. Compositions for scavenging (reducing the content of) hydrogen sulphide (H2S) or mercaptans are known. These include compositions based on alkanolamine and aldehyde reaction products such as those described in U.S. Pat. No. 4,978,512. These reaction products include triazine compounds, in particular 1,3,5 tri-(2-hydroxy-ethyl)-hexahydro-S-triazine, and are effective hydrogen sulphide scavengers; they react with hydrogen sulphide to form solids (e.g. dithiazines) which are insoluble in water. U.S. Pat. Nos. 5,347,004 and 5,554,349 describe the use of a mix of amines to react with hydrogen sulphide in natural gas systems; again, these utilise triazines.

There are problems associated with triazines. The solids are insoluble in oil and hydrocarbon/gas matrices and may cause phase separation, e.g. layers in gas processing equipment. In temperatures of 20 degrees C. or lower, solid dithiazine crystals may form in such layers, which can cause problems in processing. Other by-products of e.g. the reactions to form the triazines are environmentally unfriendly and require effective disposal. Finally, triazines do not scavenge mercaptans. There is no simple cost effective chemical being used now which will scavenge Hydrogen Sulphide, Mercaptans and Iron Sulphide all together.

### BRIEF SUMMARY OF THE INVENTION

The present technology provides systems and methods for treating a fluid with a compound comprising an azodicarbonamide or an azodicarbonamide derivative, wherein the treatment reduces the amount of hydrogen sulphide, iron sulphide and/or mercaptan in the fluid. Certain embodiments of the present technology provide a composition comprising an azodicarbonamide or an azodicarbonamide derivative and a support and/or a suspension agent.

### BRIEF DESCRIPTION OF SEVERAL VIEWS OF THE DRAWINGS

[Not Applicable]

### DETAILED DESCRIPTION OF THE INVENTION

According to the present technology in a first aspect there is provided the use, to treat a fluid (e.g. a fluid stream) to reduce the amount of one or more of hydrogen sulphide, iron sulphide and mercaptan in the fluid, of a compound comprising an azodicarbonamide or azodicarbonamide derivative.

According to the present technology in a further aspect there is provided a method for reducing the levels of one or more of hydrogen sulphide, mercaptan or iron sulphide in a fluid (e.g. a fluid stream) comprising a step of contacting the fluid (e.g. fluid stream) with a compound comprising an azodicarbonamide or azodicarbonamide derivative.

The applicants have surprisingly found that a compound comprising an azodicarbonamide or azodicarbonamide derivative may be used to reduce the levels of one or more of hydrogen sulphide, iron sulphide and mercaptans (especially hydrogen sulphide and mercaptans) in fluid (e.g. fluid streams), for example hydrocarbon, gas and water streams. The applicants' use and method (and compositions) may alleviate problems of the prior art processes associated with high concentrations of aldehydes and triazines (e.g. toxicity problems). Further, the applicants' defined use may provide very cost effective hydrogen sulphide and mercaptan scavenger compositions which may form dispersible solid by-products. The applicants' compositions, methods and uses generally utilise a simple low molecular weight compound (or compounds) which provide a good stoichiometry of mole ratios of “scavenging” (i.e. removal of hydrogen sulphide, iron sulphide and mercaptans). The scavenging reactions are fast and form generally inert products (e.g. which can be filtered off in usual crude oil or hydrocarbon systems, thereby reducing the reactions of by-products which may regenerate H2S and mercaptans down the line in crude units or petrochemical plants).

The fluid may be a fluid stream, e.g. a hydrocarbon stream (such as crude oil or natural gas) or a water stream. The hydrocarbon stream may be a gaseous stream, e.g. a sour natural gas stream. The fluid stream may be a sour water stream.

The hydrocarbon stream may be, for example, a refinery product stream, a catalytic cracker product stream, a raw gasoline stream etc. The fluid (e.g. fluid stream) may be a mixture of gas, hydrocarbon and water (e.g. a refinery product stream, which is a mixture of gas, hydrocarbon and water e.g. from the overheads of the process units). The fluid may be a crude oil. The fluid stream may be a crude oil stream containing gas, water and emulsions and mixtures thereof. The fluid may be a water, gas and hydrocarbon emulsion (e.g. such as those found in gas processing systems in oilfield gas plants and petrochemical plants).

The compound may be e.g. dissolved in an appropriate solvent, or provided in a mobile phase media or on a solid support. In one example the use (or method) is to treat a fluid (e.g. a fluid stream) to reduce the amount of hydrogen sulphide and mercaptans in the fluid (e.g. fluid stream). Preferably the stream is a hydrocarbon stream (such as crude oil or natural gas) or a water stream.

The use and/or method may reduce the level of hydrogen sulphide in the fluid to a level of 15 ppm (part per million) or less, e.g. 10 ppm or less, e.g. 5 ppm or less e.g. to substantially zero (substantially completely removed). The use and/or method may reduce the level of mercaptans in the fluid to a level of 15 ppm or less, e.g. 10 ppm or less, e.g. 5 ppm or less e.g. to substantially zero (substantially completely removed).

The treatment of the fluid (step of contacting the fluid) with the compound may be, for example, one of the following: injection (e.g. continuous injection) of the compound into the fluid stream (e.g. as a solution, suspension or slurry); providing the compound in solid form (e.g. on a solid support and/or as a capsule or “pill”)—e.g. which can be dumped in the fluid (e.g. in a tank, tank system, well or formation) to treat the fluid (e.g. a mercaptan, iron sulphide and polysulphide sludge and/or sour water at the bottom of a tank); or application of the compound into the fluid (e.g. as a solution, suspension or slurry) in a batch (e.g. by filling a batch vessel with substrate and “scavenger” compound, with e.g. subsequent removal (drain, pump, decant etc.) of treated fluid).

The compound comprising an azodicarbonamide or azodicarbonamide derivative may be, for example, a compound of formula (I):

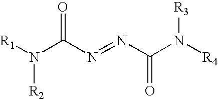

wherein:

(a) R₁, R₂, R₃ and R₄ are independently selected from a hydrogen atom, an optionally substituted (C₁-C₁₀)alkyl group, an optionally substituted (C₃-C₁₀)cycloalkyl group, an optionally substituted (C₂-C₁₀)alkenyl group, an optionally substituted (C₃-C₁₀)aryl group, and an optionally substituted (C₃-C₁₀)heterocylic group including one or more heteroatoms independently selected from N, O, S or P; or

(b) one or both of pair R₁ and R₂ and pair R₃ and R₄ represent an optionally substituted chain of between 1 and 10 atoms forming a heterocyclic group including the Nitrogen atom bound to pair R₁ and R₂ and/or pair R₃ and R_{4,} unlinked R₁, R₂, R₃, R₄ groups (if any) being selected from the groups in (a) above. Groups which are optionally substituted may be substituted by up to five groups independently selected from, for example, an —OH group, an amine group, a (C₁-C₁₀)alkyl group, a (C₃-C₁₀)cycloalkyl group, a (C₂-C₁₀)alkenyl group, a (C₃-C₁₀)aryl group, and a (C₃-C₁₀)heterocylic group.

Preferably, R₁, R₂, R₃ and R₄ are independently selected from a hydrogen atom and an optionally substituted (C₁-C₁₀)alkyl group. In one example, R₁, R₂, R₃ and R₄ are all H, and the compound of formula (I) is azodicarbonamide.

According to the present technology in a further aspect there is provided a composition comprising an azodicarbonamide or azodicarbonamide derivative; and a support. Preferably the support is a solid support. Preferably the support is a resin, e.g. an active resin. Preferably the azodicarbonamide or azodicarbonamide derivative is held in a reactive form on the resin. The support may be an inert support, e.g. clay (e.g acid washed clay), or diatomaceous earth.

The ratio (by weight) of azodicarbonamide or azodicarbonamide derivative:support may be between 1:1 and 1:10 e.g. between 1:2 and 1:7 e.g. 1:4.

The resin may be an activated resin. The resin may be an ion exchange resin, for example a cationic resin, for example a cationic macroreticular resin, for example a strongly acidic cationic resin. The resin may have a sulphonic acid functionality. The resin may be one of those sold under the trade marks Amberjet, Amberlite or Amberlyst (Aldrich) for example Amberjet™ 1200 (H) ion exchange resin ref 43,673-9; Amberlite® IR series as IR 120 (plus) ref 21, 653-4; or Amberlyst 15 (see elsewhere herein).

The composition may comprise azodicarbonamide or azodicarbonamide derivative held in a (e.g. reacted, solid) resin. The composition may be suitable for use in a column (as described below), or for use as a solid [e.g. as a pill or capsule (see below)].

The composition may comprise a matrix including the azodicarbonamide or azodicarbonamide derivative and the support (e.g. resin) [for example formed by reaction of azodicarbonamide or azodicarbonamide derivative with a resin for example a cationic macroreticular resin], and may further comprise a solvent (e.g. water, propylene glycol methyl ether (e.g. Dowanol®) or mixture thereof). The ratio of matrix:solvent (by weight) is between 1:99 and 100:0, and is preferably between 60:40 and 40:60, e.g. 50:50. The matrix:solvent composition may be a slurry, a suspension or stable dispersion. The composition may further comprise an acid or acids. The composition (e.g. if in the form of a slurry, a suspension or stable dispersion) may be particularly suitable for application to a fluid or fluid stream by injection; and/or for use in a column. The composition may be applied to a fluid stream by injection, e.g. continuous or batch or pulsed injection. Dowanol® is a trade mark of Dow Chemicals.

According to the present technology in a still further aspect there is provided a composition comprising: an azodicarbonamide or azodicarbonamide derivative; and a suspension agent.

The suspension agent may be, for example, a jelly, for example petroleum jelly. The suspension agent may be, for example, gum arabic, gum acacia, xanthan gum, a cellulose (e.g. carboxy methyl cellulose (CMC) or hydroxy ethyl cellulose (HEC)), starch, or e.g. mixtures thereof. The composition may further comprise a solvent or mixture of solvents. Preferred solvents include water, polyglycols, other alcohols such as isopropyl alcohol, or mixtures of two or more of the above. The composition may comprise, for example, azodicarbonamide or azodicarbonamide derivative; jelly; and water (optionally further comprising polyethylene glycol). The composition may comprise, for example, azodicarbonamide or azodicarbonamide derivative; CMC or HEC; and water (optionally further comprising polyethylene glycol). Preferably the ratio (by weight) of azodicarbonamide or azodicarbonamide derivative:suspension agent is between 40:1 and 1:1, e.g. 20:1 and 5:1, e.g. between 10:1 and 6:1, e.g. 8:1. The composition may be particularly suitable for application to a fluid or fluid stream by injection. The composition may be a stable dispersion or suspension. The composition may be applied to fluid stream by injection, e.g. continuous or batch or pulsed injection.

In compositions according to aspects of the present technology the azodicarbonamide or azodicarbonamide derivative may be a compound of formula (I)

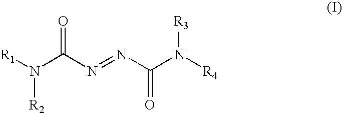

wherein

(a) R₁, R₂, R₃ and R₄ are independently selected from a hydrogen atom, an optionally substituted (C₁-C₁₀)alkyl group, an optionally substituted (C₃-C₁₀)cycloalkyl group, an optionally substituted (C₂-C₁₀)alkenyl group, an optionally substituted (C₃-C₁₀)aryl group, and an optionally substituted (C₃-C₁₀)heterocylic group including one or more heteroatoms independently selected from N, O, S or P; or

(b) one or both of pair R₁ and R₂ and pair R₃ and R₄ represent an optionally substituted chain of between 1 and 10 atoms forming a heterocyclic group including the Nitrogen atom bound to pair R₁ and R₂ and/or pair R₃ and R₄, unlinked R₁, R₂, R₃, R₄ groups (if any) being selected from the groups in (a) above. Preferably, R₁, R₂, R₃ and R₄ are independently selected from a hydrogen atom and an optionally substituted (C₁-C₁₀)alkyl group. In one example, R₁, R₂, R₃ and R₄ are all H, and the compound of formula (I) is azodicarbonamide.

Compositions according to aspects of the present technology may be suitable for application to e.g. fluid streams (containing mercaptans and/or hydrogen sulphide and/or iron sulphide), e.g. by injection, by contact of the fluid/fluid streams on a column loaded with the composition in, for example, a resin (e.g. Amberlyst 15 ® resin), or in pill or encapsulated forms. When a column is used, the scavenged by-products on the column and unused (scavenger) composition may be removed e.g. by purging with steam which can break up the unused chemical to nitrogen, carbon dioxide and ammonia-gases common in refineries. The by-products can be purged to sour gas strippers which deal with mercaptans, ammonia and hydrogen sulphides. Rapid quenching with water will convert the carbon dioxide to carbonic acids and ammonia to salts. Amberlyst 15 ® is a trade mark of Rohm and Haas Company.

In compositions, methods and uses according to the present technology a solvent may be used as described above. As well as solvents mentioned above the solvent may be for example an alkane (e.g. a C2 to C15 branched or straight chain alkane such as hexane, pentane); an alcohol (e.g. a C1 to C15 branched or straight chain alcohol); a glycol (e.g. polyethylene glycol or other glycol e.g. ethylene, propylene or diethylene glycol or the higher polyalkylene homologues of these e.g. polypropylene glycols or mixtures thereof); or an aromatic solvent (e.g. toluene, xylene etc.); or mixtures of one or more of the solvents mentioned above. Other solvents may be used as is readily understood by the person skilled in the art.

Embodiments of the present technology will now be described in more detail.

### EXAMPLE 1

### Suspension

Azodicarbonamide (ADC), a solid, obtained from Aldrich, ref number A9, 660-6, 97% purity, is mixed with petroleum jelly, polyethylene glycol, and water in the ratio by weight ADC:petroleum jelly:polyethylene glycol:water of 40:5:20:35. The resulting composition is a suspension of the ADC (because the jelly increases the viscosity of the mixture enabling the solid ADC to remain suspended). The inclusion of polyethylene glycol renders the Example 1 composition particularly suitable for use in cold climates. The polyethylene glycol can be substituted with other glycols e.g. ethylene, propylene or diethylene glycol or the higher polyalkylene homologues of these e.g. polypropylene glycols or mixtures thereof.

### EXAMPLE 2

### Suspension

Azodicarbonamide (ADC), a solid, obtained from Aldrich, ref number A9, 660-6, as above, is mixed with petroleum jelly and water in the ratio by weight ADC:petroleum jelly:water of 40:5:55. The product is a suspension of the ADC.

### EXAMPLES 2a AND 2a′

### Suspensions

Azodicarbonamide (ADC), a solid, obtained from Aldrich, ref number A9, 660-6, as above, is mixed with carboxymethyl cellulose (CMC) and water in the ratio by weight ADC:CMC:water of 25:2:73. The product is a suspension of the ADC. A further example (Example 2a′) was obtained by mixing ADC with carboxymethyl cellulose (CMC) and water in the ratio by weight ADC:CMC:water of 25:1:74. The product is a suspension of the ADC. It will be appreciated that glycols (e.g. PEG) and or Dowanol® etc. may be added e.g. to winterise the examples.

### EXAMPLE 3

### Liquid-Slurry for Injection

The Azodicarbonamide (ADC) is prepared on a resin in a beaker or bottle, as follows. A strongly cationic macroreticular resin (10 g resin obtained as Amberlyst 15® code A-15-Kation #1, from Aldrich) is suspended in 50 ml distilled water. The resin specifications are as follows: ref: 15635.0500 Merck, particle size>95% of (0.355-1.18 mm diameters), capacity>1.7 mmol/ml. The resin is stirred and washed (2-3 times) to remove impurities from manufacturing until the washings are clear, white and colourless. The washed resin is stirred with 50 ml of 5% aqueous sulphuric acid to activate it, as is well known in the art; after 2 minutes, the spent acid is decanted. The acid activated resin is now washed with distilled water (50 ml portions) until the pH of water is near 6-6.5. The resin is drained of free water.

10 g ADC is added to the resin, together with 5 ml of water (at 45 deg C.) and shaken (e.g in a capped bottle). A further 5 ml water is added and the mixture stirred (or shaken) at 45 deg C. until a slurry is obtained. Unslurried water is then decanted off and the slurry is ready for use by e.g. injection.

### EXAMPLE 4

### Injection

The compositions of Examples 1, 2, 2a, 2a′ and 3 are suitable for injection to liquid streams. The composition is injected (as a suspension or slurry) into liquid or gas or water stream by techniques well known in the art. A notched quill atomizer system may be used, particularly for gaseous streams. The stream (and scavenging) is monitored by the appropriate analytical techniques such as electrometric titration of liquids or by Drager tube detection of Hydrogen sulphide and mercaptans for gas.

### EXAMPLE 5

### Preparation of a Solid Support Column

A column is fitted with sintered metal discs to stop ADC eluting off the column in solid form (as is well known in the art). The Azodicarbonamide (ADC) is prepared on a resin, e.g. a strongly cationic macroreticular resin (although an inert solid support, for example, diatomaceous earth, acid washed clays etc. may also be used) in an ion exchange column with a heated jacket to maintain the column temperature at 45 degrees C. (resin obtained as Amberlyst 15® code A-15-Kation #1, from Aldrich). The resin specifications are as follows: ref: 15635.0500 Merck, particle size>95% of (0.355-1.18 mm diameters), capacity>1.7 mmol/ml. The resin is washed in batch vessels to remove impurities from manufacturing until the washings are clear, white and bright. The washed resin is contacted with 1-10% aqueous hydrochloric or sulphuric acid to activate it, as is well known in the art; after a certain time, the spent acid is decanted. The acid activated resin is now washed to a pH of 6.5 with distilled water. The column is run dry of free water. The weight of ADC is chosen at weight to resin ratio of 45:55. The ADC (1.164 g ADC in 10 ml zylene) is added to the column, and the ADC washed through with water—allowing it to react with the resin beads.

For column work, the fluids (e.g. oil) to be treated are eluted down the column and tested periodically as in Example 4 above to determine the degree of scavenging and determination of when the column is being spent (i.e. scavenging is decreasing because the ADC is almost completely reacted). The treated fluids are, for example, filtered and sent to storage and monitored by the analytical techniques described above.

Once the column is spent, the fluid is switched to another column(s) and the spent resin (with by-products on the column) is purged with steam, to break down by-products and spent ADC. The broken down by-products may, for example, be sent to sour gas strippers. The preferred purging method is using steam but other chemical purging or regeneration methods can be used.

Alternatively, a solid support resin may be stirred in water in a batch vessel with ADC. The prepared ADC on the solid support formed in batch vessels in this way is transferred to the column in a slurry and washed as above, until the drained liquids are clear/colourless.

### EXAMPLE 6

### An ADC Pill and Encapsulation

The ADC can be prepared in an “inert pill” or as a capsule by standard manufacturing/encapsulation techniques. Encapsulated and pill products may for example be sent “downhole” through tubing to the bottom of the tubing (e.g. ‘rat hole’) or can be placed in gravel packs, for example, and following slow release of ADC (from the capsule or pill), the scavenging takes place. Pill products may be used in tanks; the pill(s) is dropped into the tank and then (allowing sufficient time for the pill to react), the tank contents are filtered. Treated fluids are monitored as in Example 4 above. Samples of the tank bottoms for testing can be taken by collecting thief samples or via the stopcocks.

### EXAMPLE 7

### Demonstration of Hydrogen Sulphide and Mercaptan Scavenging

The following tests demonstrate effective scavenging of H2S and mercaptan using compositions which are examples of the present technology.

### A Composition for Testing

A dispersion of ADC (0.5 g) in 0.5 grams petroleum jelly and 1.5 grams polyethylene glycol was made by the method of Example 1 above (without the water).

### H2S Scavenger Method

### Test 1

A 5 ml test sample of water containing H2S and iron sulphide was taken and the scavenger dispersion was added in an amount equal to 10 ppm ADC. The mixture was shaken at room temp 20 deg C., then heated and H2S measured by the colour method (e.g. the Palin's hydrogen sulfide photometric method, as is well known in the art). The test sample was compared with a 5 ml test sample of water containing H2S and iron sulphide as a control “BLANK”. At Time zero, BLANK had a H2S amount of 103 ppm. After heating for 1 hr at 57 deg C., BLANK has 89 PPM H2S detected; the test sample (with ADC scavenger) has 7.8 ppm H2S. After heating for 2 hr at 57 deg C., BLANK has 58 PPM H2S detected; the test sample (with ADC scavenger) has 0 ppm H2S; i.e. none detected. This demonstrates effective scavenging (removal) of H2S by a composition of the present technology.

### Test 2

Two 5 ml test samples of water containing H2S and iron sulphide were taken and 10 ppm of ADC [either scavenger dispersion preparation “ADC1” or “ADC2” (both prepared by the method described in “composition for testing” above) are added, each sample−containing ADC 1 or ADC 2—was mixed/shaken as above. (ADC-1 was obtained from Aldrich as per ref above, ADC-2 was obtained as AZOBUL®B from Arkema Chemicals, Division Oxygenes, Puteaux, France. AZOBUL® is a trademark of Arkema). The test samples were compared with a 5 ml test sample of water containing H2S and iron sulphide as a control “BLANK”. At Time zero, BLANK had a H2S amount of 100 ppm. After 30 min at room temperature, BLANK has 93.7 PPM H2S detected; the ADC1 test sample has 2.92 ppm H2S; the ADC 2 test sample has 28 ppm H2S. This demonstrates effective scavenging of H2S by compositions of the present technology. The difference between ADC 1 and ADC 2 was the chemical grade (i.e. ADC 1 was a better chemical grade). (It was noticeable that the black colour due to iron sulphide solids being present disappeared)

The following Tests were performed using the ADC 2 grade. Thus it is expected that improved results would be provided if the experiments were repeated with higher grade ADC.

### Test 3

Samples were prepared as described above. At Time zero, BLANK had a H2S amount of 132 ppm. After heating for 30 min at 55 deg C., a test sample (with ADC concentration 50 ppm) has 12 ppm H2S. After heating for 30 min at 55 deg C., a test sample (with ADC scavenger conc. 50 ppm) has 18 ppm H2S. After heating for 1 hour at 55 deg C., a test sample (with ADC concentration 12.5 ppm) has 3 ppm H2S (Blank has H2S 95 ppm). Again, this demonstrates effective scavenging of H2S by a composition of the present technology.

### Test 4

Samples were prepared as described above. After heating at 55 deg C. for 15min, BLANK had a H2S amount of 56 ppm. After heating for 15 min at 55 deg C., a test sample (with ADC concentration 12.5 ppm) has 24 ppm H2S. A duplicate repeat test sample (with ADC concentration 12.5 ppm) has 26 ppm H2S. After heating for 15 min at 55 deg C., a test sample (with ADC concentration 25 ppm) has 14 ppm H2S. A duplicate repeat test sample (with ADC concentration 25 ppm) has 12 ppm H2S. Again, this demonstrates effective scavenging of H2S by a composition of the present technology.

### Test 5: H2S Scavenging in Mercaptan Crude Oil

Testing methods were similar to those above, and are well known in the art. A sample CRUDE OIL 1 had a measured value H2S of 36 ppm at time Zero. A sample CRUDE OIL 2 had a measured value H2S of 17 ppm at time Zero. 20 ppm of the ADC 2 scavenger dispersion (i.e. including 20 ppm ADC) were added to each sample and heated at 45 deg C. for 15 minutes. Crude oil 1 gives 3 ppm residual H2S after 15 min; Crude oil 2 gives 1.1 ppm residual H2S at this time. This demonstrates effective scavenging of H2S in crude oil by a composition of the present technology.

### Test 6: Mercaptan Scavenging in Crude Oils

Testing methods are well known in the art—e.g. the Electometric KOH or silver nitrate titration methods were used to assess the amount of mercaptans. The amount of mercaptan present in untreated Crude Oil 1 was measured by the amount of RSH neutralizer required; this was shown to be 0.8 mg/g oil. The amount of RSH neutralizer required for Crude Oil 2 was shown to be 0.57 mg/g oil. A 200 ppm dose of ADC 2 in the scavenger dispersion was added to each sample and heated at 45 deg C. for 15 minutes. After this time Crude oil 1 required RSH neutralizer in an amount of 0.2 mg/g oil; Crude oil 2 required RSH neutralizer in an amount 0.12 mg/g oil. Less neutralizer was required for both oils following treatment with 200 ppm ADC composition, clearly indicating a reduction in mercaptans. Thus, the addition of the scavenger composition reduces the mercaptans in crude oils 1 and 2.

Test 7: Mercaptan Scavenging in Crude Oils (column Method)

ADC (5 grams of ADC on 15 grams of resin) was prepared on a solid support A15 resin by the method of Example 5 above.

### Test 8

Testing methods are well known in the art. The amount of mercaptan present in untreated Crude Oil 1 was measured by the amount of RSH neutralizer required; this was shown to be 0.8 mg/g oil. The amount of RSH neutralizer required for Crude Oil 2 was shown to be 0.57 mg/g oil. About 7.1 to 7.8 grams of each crude were eluted through a column containing the 100 ppm ADC/resin at a temperature of 20 deg C. After elution Crude oil 1 required RSH neutralizer in an amount of 0.16 mg/g oil; Crude oil 2 required RSH neutralizer in an amount of 0.09 mg/g oil. Thus, the ADC scavenger held on a support resin reduces the mercaptan amount in crude oils 1 and 2.

### Test 9: Mercaptan Scavenging in Crude Oils (Injection Method)

Testing methods are well known in the art. The amount of mercaptan present in untreated Crude Oil 1 was measured by the amount of RSH neutralizer required; this was shown to be 0.8 mg/g oil. About 7.1 to 7.9 grams of oil were treated with 100 ppm ADC scavenger dispersion prepared as outlined in “composition for testing” above at a temperature of 45 deg C. for 15 and 30 min. After 15 min Crude oil 1 required RSH neutralizer in an amount of 0.34 mg/g oil; after 30 min Crude oil 1 required RSH neutralizer in an amount of 0.14 mg/g oil. Thus, injection of the scavenger reduces the mercaptan amount in crude oils 1 and 2.

The present technology has now been described in such full, clear, concise and exact terms as to enable a person familiar in the art to which it pertains, to practice the same. It is to be understood that the foregoing describes preferred embodiments and examples of the present technology and that modifications may be made therein without departing from the spirit or scope of the present technology as set forth in the claims. Moreover, while particular elements, embodiments and applications of the present technology have been shown and described, it will be understood, of course, that the present technology is not limited thereto since modifications can be made by those familiar in the art without departing from the scope of the present disclosure, particularly in light of the foregoing teachings and appended claims. Moreover, it is also understood that the embodiments shown in the drawings, if any, and as described above are merely for illustrative purposes and not intended to limit the scope of the present technology, which is defined by the following claims as interpreted according to the principles of patent law, including the Doctrine of Equivalents. Further, all references cited herein are incorporated in their entirety.

1. A method of treating a fluid to reduce the amount of at least one of hydrogen sulphide, iron sulphide and mercaptan in the fluid comprising treating a fluid with a compound comprising an azodicarbonamide or an azodicarbonamide derivative.

2. The method of claim 1 wherein the fluid is a fluid stream.

3. The method of claim 1 wherein said compound is injected into the fluid.

4. The method of claim 1, wherein said compound is in solid form when in contact with the fluid.

5. The method of claim 1 wherein the compound comprising an azodicarbonamide or azodicarbonamide derivative is a compound of formula (I) 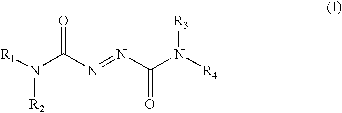
wherein R₁, R₂, R₃ and R₄ are independently selected from the group consisting of a hydrogen atom, an optionally substituted (C₁-C₁₀)alkyl group, an optionally substituted (C₃-C₁₀)cycloalkyl group, an optionally substituted (C₂-C₁₀)alkenyl group, an optionally substituted (C₃-C₁₀)aryl group, and an optionally substituted (C₃-C₁₀)heterocylic group including one or more heteroatoms independently selected from N, O, S or P.

6. The method of claim 1 wherein the compound comprising an azodicarbonamide or azodicarbonamide derivative is a compound of formula (I) 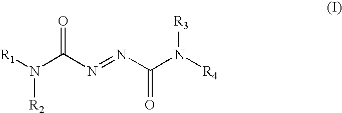
wherein one or both of pair R₁ and R₂ and pair R₃ and R₄ represent an optionally substituted chain comprising between 1 and 10 atoms forming a heterocyclic group including the Nitrogen atom bound to at least one of pair R₁ and R₂ and pair R₃ and R₄, and wherein the compound comprises unlinked R₁, R₂, R₃, R₄ groups, said unlinked R₁, R₂, R₃ and R₄ groups are independently selected from the group consisting of a hydrogen atom, an optionally substituted (C₁-C₁₀)alkyl group, an optionally substituted (C₃-C₁₀)cycloalkyl group, an optionally substituted (C₂-C₁₀)alkenyl group, an optionally substituted (C₃-C₁₀)aryl group, and an optionally substituted (C₃-C₁₀)heterocylic group including one or more heteroatoms independently selected from N, O, S or P.

7. The method of claim 5, wherein R₁, R₂, R₃ and R₄ are independently selected from a hydrogen atom and an optionally substituted (C₁-C₁₀)alkyl group.

8. The method of claim 6, wherein R₁, R₂, R₃ and R₄ are independently selected from a hydrogen atom and an optionally substituted (C₁-C₁₀)alkyl group.

9. A method for reducing the levels of one or more of hydrogen sulphide, mercaptan or iron sulphide in a fluid comprising a step of contacting the fluid with a compound comprising an azodicarbonamide or azodicarbonamide derivative.

10. A composition comprising an azodicarbonamide or an azodicarbonamide derivative and a support.

11. The composition of claim 10, wherein said support is a solid support.

12. The composition of claim 10, wherein said support is a resin.

13. The composition of claim 10, wherein the ratio of the azodicarbonamide or the azodicarbonamide derivative to the support, by weight, is between 1:1 and 1:10.

14. The composition of claim 10, further comprising a matrix including the azodicarbonamide or azodicarbonamide derivative and the support.

15. The composition of claim 14, further comprising a solvent.

16. The composition of claim 10, wherein the composition is in the form of at least one of a slurry, a suspension dispersion, a stable dispersion, or for use in a column.

17. The composition of claim 10, wherein the azodicarbonamide or the azodicarbonamide derivative is a compound of formula (I) 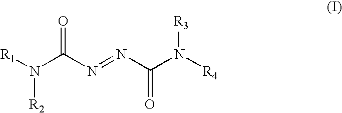
wherein R₁, R₂, R₃ and R₄ are independently selected from the group consisting of a hydrogen atom, an optionally substituted (C₁-C₁₀)alkyl group, an optionally substituted (C₃-C₁₀)cycloalkyl group, an optionally substituted (C₂-C₁₀)alkenyl group, an optionally substituted (C₃-C₁₀)aryl group, and an optionally substituted (C₃-C₁₀)heterocylic group including one or more heteroatoms independently selected from N, O, S or P.

18. The composition of claim 10, wherein the azodicarbonamide or the azodicarbonamide derivative is a compound of formula (I) 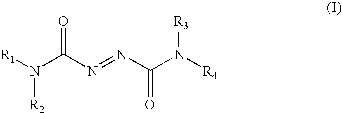
wherein one or both of pair R₁ and R₂ and pair R₃ and R₄ represent an optionally substituted chain comprising between 1 and 10 atoms forming a heterocyclic group including the Nitrogen atom bound to at least one of pair R₁ and R₂ and pair R₃ and R₄, wherein the compound comprises unlinked R₁, R₂, R₃, R₄ groups, said unlinked R₁, R₂, R₃ and R₄ groups are independently selected from the group consisting of a hydrogen atom, an optionally substituted (C₁-C₁₀)alkyl group, an optionally substituted (C₃-C₁₀)cycloalkyl group, an optionally substituted (C₂-C₁₀)alkenyl group, an optionally substituted (C₃-C₁₀)aryl group, and an optionally substituted (C₃-C₁₀)heterocylic group including one or more heteroatoms independently selected from N, O, S or P.

19. A composition comprising an azodicarbonamide or an azodicarbonamide derivative and a suspension agent.

20. The composition of claim 19 wherein the suspension agent is a jelly.

21. The composition of claim 19 further comprising a solvent.

22. The composition of claim 19 wherein the ratio of the azodicarbonamide or the azodicarbonamide derivative to the suspension agent, by weight, is between 40:1 and 1:1.

23. The composition of claim 19, wherein the azodicarbonamide or the azodicarbonamide derivative is a compound of formula (I) 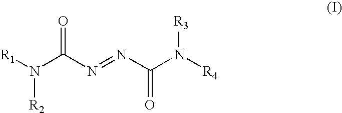
wherein R₁, R₂, R₃ and R₄ are independently selected from the group consisting of a hydrogen atom, an optionally substituted (C₁-C₁₀)alkyl group, an optionally substituted (C₃-C₁₀)cycloalkyl group, an optionally substituted (C₂-C₁₀)alkenyl group, an optionally substituted (C₃-C₁₀)aryl group, and an optionally substituted (C₃-C₁₀)heterocylic group including one or more heteroatoms independently selected from N, O, S or P.

24. The composition of claim 19, wherein the azodicarbonamide or the azodicarbonamide derivative is a compound of formula (I) 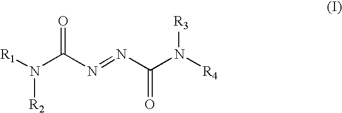
wherein one or both of pair R₁ and R₂ and pair R₃ and R₄ represent an optionally substituted chain comprising between 1 and 10 atoms forming a heterocyclic group including the Nitrogen atom bound to at least one of pair R₁ and R₂ and pair R₃ and R₄, and wherein the compound comprises unlinked R₁, R₂, R₃, R₄ groups, said unlinked R₁, R₂, R₃ and R₄ groups are independently selected from the group consisting of a hydrogen atom, an optionally substituted (C₁-C₁₀)alkyl group, an optionally substituted (C₃-C₁₀)cycloalkyl group, an optionally substituted (C₂-C₁₀)alkenyl group, an optionally substituted (C₃-C₁₀)aryl group, and an optionally substituted (C₃-C₁₀)heterocylic group including one or more heteroatoms independently selected from N, O, S or P.

25. The composition of claim 19, further comprising:
a) an azodicarbonamide or an azodicarbonamide derivative;
b) at least one of the following: jelly, CMC and HEC; and
c) water.

26. The composition of claim 25 further comprising polyethylene glycol.
